(19) United States
(12) Patent Application Publication
(10) Pub. No.: US 2008/0299098 A1
(43) Pub. Date: Dec. 4, 2008

(54) Broad-Spectrum Antibacterial and Antifungal Activity of Lactobacillus Johnsonii D115

(76) Inventors: Chea-Yun Se, Pontian Johor (MY); Fui-Fong Yong, Singapore (SG); Hai-Meng Tan, Bukit Regency (SG); Wee Ming Yeo, Singapore (SG); Alex Yeow-Lim Teo, Minton Rise Condominium (SG)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(21) Appl. No.: 12/109,159

(22) Filed: Apr. 24, 2008

Related U.S. Application Data
(60) Provisional application No. 60/925,937, filed on Apr. 24, 2007.

(51) Int. Cl. A61K 35/74; C12N 1/20; A61P 31/04

(52) U.S. Cl. 424/93.45; 435/252.9

(57) ABSTRACT

The present invention demonstrated the potential use of Lactobacillus johnsonii D115 as a probiotic, as a prophylactic agent or as a surface treatment of materials against human and animal pathogens such as Brachyspira pilosicoli, Brachyspira hyodysenteriae, Shigella sonnei, Vibrio cholera, Vibrio parahaemolyticus, Campylobacter jejuni, Streptococcus pneumoniae, Enterococcus faecalis, Enterococcus faecium, Clostridium perfringens, Yersinia enterocolitica, Escherichia coli, Klebbsiella pneumoniae, Staphylococcus aureus, Salmonella spp., Bacillus cereus, Aspergillus niger and Fusarium chlamydosporum. The proteineous antimicrobial compound was partially characterized and found to be heat tolerant up to 121° C. for 15 min, and acid tolerant up to pH1 for 30 min at 40° C. The compound is also stable to enzymatic digestion, being able to retain more than 60% antimicrobial activity when treated with pepsin and trypsin.

### RELATED APPLICATIONS

This application claims priority to U.S. Patent Application Ser. No. 60/925,937, filed Apr. 24, 2007, and incorporated herein by this reference.

### BACKGROUND OF THE INVENTION

The present invention relates generally to bacteria having antimicrobial activity and, more specifically, to bacteria of Lactobacillus johnsonii that has both antibacterial and antifungal activity, and including Lactobacillus johnsonii strain D115.

The genus Brachyspira (formerly Treponema and Serpulina) consists of several species such as Brachyspira innocens, B. murdochii, B. intermedia, B. hyodysenteriae and B. pilosicoli. These bacteria are Gram-negative spirochetes (loosely-coiled morphology), motile, oxygen tolerant and anaerobes with hemolytic activity on blood agar. Among all, B. hyodysenteriae and B. pilosicoli are of considerable importance due to their high pathogenicity in causing severe diarrhoeal disease and poor growth rates in various animal species, resulting in substantial productivity and economic losses. In pigs, B. hyodysenteriae and B. pilosicoli are respectively the etiologic agents of swine dysentery and porcine intestinal spirochetosis. Despite being of the same genus, B. hyodysenteriae and B. pilosicoli differ in their hemolytic activity which clearly distinguish the colonic disease caused by each of the spirochaetes.

Swine dysentery is a highly contagious diarrhea disease that can occur in pigs of all ages with higher incidence observed in growing and finishing pigs. The first description of swine dysentery was in 1921 and with the etiological agent, Treponema hyodysenteriae, clearly elucidated in 1971. The disease is a muco-haemorrhagic colitis, characterized by inflammation, excess mucus production, and necrosis of the mucosa layer of large intestines. Pigs infected by the causative agent, B. hyodysenteriae, will show clinical signs such as weight loss, depression, reduced appetite, and most notably the change in the feces appearance to a dark brown color (start of swine dysentery) and bloody diarrhea (severe stage) due to the strong beta-hemolytic activity of B. hyodysenteriae. Death usually results from the prolonged dehydration due to severe diarrhea. In the case when recovery of infected pigs is possible, the pigs have slow growth rates and most importantly, could harbor the organism and risk passing the infection to other pigs. The occurrence of swine dysentery has been reported in several countries such as Australia, Italy, German, Switzerland, Denmark, United States (US), United Kingdom (UK) and Czech Republic. In the United Kingdom, prevalence of swine dysentery in pigs was estimated to be around 11% of herds. In Australia, it has been estimated that $100 per sow per year is lost to the disease while the annual losses due to the disease was estimated to be as much as $115.2 million in the US. The significant economic loss due to swine dysentery is contributed by the cost of medication, additional animal care, reduced animal growth rates, reduced feed conversion efficiencies and high mortality rate.

Compared to swine dysentery, porcine intestinal spirochetosis (PIS) is a non-fatal and milder form of diarrheal disease caused by the weakly beta-hemolytic B. pilosicoli. The disease commonly occurs in weaner and grower pigs between 4 and 20 weeks. The clinical signs associated with this disease include mucus-containing and non-bloody diarrhea, poor feed conversion and depressed growth rate. The occurrence of PIS has been reported in several countries such as the United Kingdom, Australia, Brazil and Sweden. In a recent survey in the United Kingdom, B. pilosicoli was reported to be responsible for colitis in 44 out of 85 pig unit. In the study in Brazil⁸, B. pilosicoli was identified as the agent in causing diarrhea in pigs in 7 out of 17 farms. Apart from swine, B. pilosicoli is also implicated in causing disease in human, dogs and birds. In chickens, infection with the pathogenic spirochaetes has been termed Avian Intestinal Spirochaetosis (AIS) and has been receiving much attention in Australia.

The transmission and infection route of Brachyspira spp. is primarily due to ingestion of fecal material from infected animals⁴¹. The spread of the disease is further aided when fecal material is moved through contaminated boots and vehicles; or into drinking water of animals⁴⁸. Studies have demonstrated the survivability of B. hyodysenteriae and B. pilosicoli in porcine feces at 10° C. and up to 112 and 210 days, respectively. An early study showed that B. hyodysenteriae was viable in dysenteric pig feces up to 1 and seven days at 37 and 25° C., respectively. The first sign of swine dysentery was reported to be 5-10 days after pigs were infected by the organism^{23,29}. The incubation period of diarrhea disease caused by B. pilosicoli was found to be 4-9 days⁵², and between 9 and 24 days in a more recent study²⁵. The pathogenecity and diarrhea-causing ability of these bacteria lie with the association with intestinal mucosa although the exact mechanism of association has not been completely elucidated. Brachyspira hyodysenteriae was shown to have a chemotactic response towards mucus and is high motility in mucus compared to other intestinal bacteria, which facilitates penetration into mucosa where hemolysin can be released, which is an important factor in the pathogenesis of the disease^{22,28,29,37}. Presence of hemorrhage, fibrin, mucus, edema, necrosis and hyperemia are the common macroscopic signs of B. hyodysenteriae infection in the colon²². In contrast to disease severity, gross lesions caused by B. pilosicoli are relatively milder with greenish to greenish-gray colonic content and without evidence of blood or increased mucus production²⁵. Brachyspira pilosicoli colonizes large intestines through end-on attachment to the luminal epithelium, forming a false brush border of spirochetes cells which differs from no specific attachment of B. hyodysenteriae²⁵.

Treatment and control of diarrhea diseases caused by Brachyspira spp. rely heavily on the use of antibiotics such as tylosin, tiamulin, lincomycin, gentamicin and valnemulin^{14,41}. However, the use of antibiotics has always been revolving around the issue of development of antimicrobial resistance in Brachyspira spp. Several studies reported on the increased resistance of strains of Brachyspira spp to tylosin, lincomycin, tetracycline and gentamicin^{14,16,19,27,41,49}. In addition, the high genetic diversity of strains of Brachyspira spp. found in animals further complicates the problem⁴².

Shigellosis accounts for more than 300,000 cases annually worldwide and fatality may be as high as 10-15% with some strains. However, this disease occurs rarely in animals; it is principally a disease of human and other primates such as monkeys and chimpanzees. Outbreaks due to Shigella infection are difficult to control because of their low infectious dose. Increased numbers of cases in a community that appear to be sporadic may in fact be due to unrecognized outbreaks. Shigellosis is caused by any of the four species of Shigella, namely Shigella dysenteriae, Shigella flexneri, Shigella boydii and Shigella sonnei. Of these, Shigella sonnei is the most prevalent (77%) species in industrialized countries and the second most prevalent in developing countries, followed by Shigella flexneri. Some strains have been known to produce enterotoxin and Shiga toxin¹¹. The organism is frequently found in water polluted with human feces and food products like salads (potato, tuna, shrimp, macaroni, and chicken), raw vegetables, milk and dairy products, and poultry can be contaminated through the fecal-oral route.

The genus Vibrio consists of Gram-negative straight or curved rods, motile by means of a single polar flagellum. It is one of the most common organisms in surface waters of the world. They occur in both marine and freshwater habitats and in association with aquatic animals. Some species are bioluminescent and live in mutualistic associations with fish and other marine life. Other species are pathogenic for fish, eels, frogs and primates. V. cholerae and V. parahaemolyticus are pathogens of human. Both produce diarrhea, but in ways that are entirely different. V. parahaemolyticus is an invasive organism affecting primarily the colon; V. cholerae is noninvasive, affecting the small intestine through secretion of an enterotoxin¹¹. The infection is often mild or without symptoms, but sometimes it can be severe. Approximately one in 20 infected persons has severe disease characterized by profuse watery diarrhea, vomiting, and leg cramps. In these persons, rapid loss of body fluids leads to dehydration and shock. Without treatment, death can occur within hours. Cholera diarrhea is one of three diseases requiring notification to WHO under the International Health Regulations due to its long epidemic history. For example, in 1994 in a refugee camp in Goma, Democratic Republic of the Congo, a major epidemic took place. An estimated 58 000-80 000 cases and 23 800 deaths occurred within one month. Similarly, in 1961 in Sulawesi, Indonesia, the disease spread rapidly to other countries in Asia, Europe and Africa and finally to Latin America in 1991 causing nearly 400 000 reported cases and over 4000 reported deaths that year. The yearly estimate of cases was 400,000 and the yearly estimate of deaths was 5,000.

Before the 1990s, it was thought that vancomycin-resistant enterococci were present only in hospitals where vancomycin had been used for many years. However, it has become increasingly evident that vancomycin-resistant enterococci are easily recovered from farm animals that are fed avoparcin^{1,9,30}. Although Enterococcus faecalis is a more common cause of disease in human, resistance to vancomycin is more frequent among E. faecium isolates. As part of the Danish program of monitoring for antimicrobial resistance from 1995 to 2000, a total of 673 Enterococcus faecium and 1,088 Enterococcus faecalis isolates from pigs together with 856 E. faecium isolates from broilers were isolated and tested for susceptibility to four classes of antimicrobial agents used for growth promotion. It was found that erythromycin resistance among E. faecium isolates from broilers reached a maximum of 76.3% in 1997 but decreased to 12.7% in 2000 concomitantly with limited usage of the drug. Use of virginiamycin increased from 1995 to 1997 and was followed by an increased occurrence of virginiamycin resistance among E. faecium isolates in broilers, from 27.3% in 1995 to 66.2% in 1997. In January 1998 the use of virginiamycin was banned in Denmark, and the occurrence of virginiamycin resistance decreased to 33.9% in 2000. Use of avilamycin increased from 1995 to 1996 and was followed by an increase in avilamycin resistance among E. faecium isolates from broilers, from 63.6% in 1995 to 77.4% in 1996.

Streptococcus pneumoniae is a Gram-positive encapsulated diplococcus. Based on differences in the composition of the polysaccharide (PS) capsule, 90 serotypes have been identified¹⁸. This capsule is an essential virulence factor. S. pneumoniae is a normal inhabitant of the human upper respiratory tract. The bacterium can cause pneumonia, usually of the lobar type, paranasal sinusitiss and otitis media, or meningitis, which is usually secondary to one of the former infections. It also causes osteomyelitis, septic arthritis, endocarditis, peritonitis, cellulitis and brain abscesses. Until 2000, S. pneumoniae infections caused 60,000 cases of invasive disease each year and up to 40% of these were caused by pneumococci non-susceptible to at least one drug. These figures have decreased substantially following the introduction of the pneumococcal conjugate vaccine for children. In the year 2002, there were 37,000 cases of invasive pneumococcal disease. Of these, 34% were caused by pneumococci non-susceptible to at least one drug and 17% were due to a strain non-susceptible to three or more drugs (CDC). Death occurs in 14% of hospitalized adults with invasive disease and transmission can occur from person to person. Based on available data, S. pneumoniae is estimated to kill annually close to one million children under five years of age worldwide, especially in developing countries where pneumococcus is one of the most important bacterial pathogens of early infancy (WHO). S. pneumoniae is not a strict human pathogen; it is known to also colonize the nasopharynx and cause respiratory disease and meningitisin several animal species.

The Campylobacteriaceae family comprises Gram-negative microaerophilic bacteria that are important zoonotic pathogens worldwide. The two most important species implicated in food-borne infections of human are C. jejuni and C. coli. Campylobacters are the leading cause of bacterial diarrhea worldwide with an estimated 1% of the Western Europe population being infected, and a key public health concern in New Zealand where the incidence rate is reportedly 370 per 100,000²¹. Typical symptoms include bloody diarrhea, abdominal pain, fever, nausea, malaise and, rarely, vomiting. In the longer term, infection with C. jejuni can lead to Guillain-Barre and Miller Fischer Syndromes³⁸. Treatment of campylobateriosis with antibiotics can reportedly lead to increasing antimicrobial resistance. Campylobacteriaceae are found in a wide range of animals, with some causing infections of the alimentary tract and reproductive tract in poultry, pigs, cattle, sheep, cats, dogs, birds, mink, rabbits and horses. The animals are thought to acquire the bacteria by contact with a contaminated environment such as water. Poultry is a major source of campylobacters with the greatest risk to human health posed by contaminated chicken. Certain foods, such as raw chicken meat, can have extremely high campylobacter counts (>10⁷ cells per carcass)²⁶. There is thus an urgent need to reduce both the incidence and levels of carcass contamination. Strict biosecurity measures have helped to control campylobacter incidence in housed birds somewhat in Scandinavia, although it remains to be seen how successful such measures can be in other parts of the world with different climates and a larger poultry industry. In contrast, a healthy and balanced gut microflora or the condition of eubiosis, is critical for the protection of animals against challenge by enteric pathogens such as campylobacters. The introduction of either beneficial probiotic bacteria or the bioactive molecules they produce that are specific against the bacteria can be an effective control measure of campylobacteriosis in both animals and human, as well as in eradicating campylobacters in fresh produce and food.

Filamentous molds and yeasts are common spoilage organisms of food and feed products, as well as stored crops and feed such as hay and silage. Moreover, food and feed products contaminated with fungi harbors potential contamination by mycotoxins^{2,44}. Similarly, animal feeds can potentially become contaminated during harvesting, processing at the feed mill or during storage, with foodborne Salmonella. Any environment that comes in contact with feed during these stages that also harbors the contaminant can theoretically contaminate the feed. This also holds true for ingredients that are combined with feeds as they are being mixed at the feed mill. Animal feeds are also potential reservoirs for cross contamination from Salmonella containing vectors and environmental sources while being fed to animals³⁶. Under conditions that are particularly conducive to mold growth such as, immature or wet crop, damaged grain, and suboptimal storage conditions such as high heat or humidity, the use of mold and bacteria inhibitors becomes necessary. Currently, available treatments and controls of Salmonella and mold growth in agricultural feeds rely heavily on the use of organic acids like propionic and formic. Food and feed preservation using anti-microbial bacteria is well documented. However, there is no documentation of the usage of bacteria for food and feed preservation against both bacterial and fungal contamination. The current invention demonstrates the possibility of prevention and treatment of food and feed against bacterial and fungal contamination. Moreover, the present invention is applicable to both surface and in vivo prevention and treatment against both human and animal pathogens.

### SUMMARY OF THE INVENTION

The invention consists of bacteria that have both antibacterial and antifungal activity. The bacteria are Lactobacillus spp. and include bacterial cells of the genus Lactobacillus species johnsonii that produce an antimicrobial metabolite(s) that is heat stable throughout the range from ambient (about 20° C.) up to at least 121° C. for at least 15 min and is acid-tolerant throughout the range from neutral to pH 1 for at least 30 min. The bacteria are preferably of strain Lactobacillus johnsonii D115.

The bacteria of the present invention have a broad-spectrum in vitro antibacterial activity against both gram positive and gram negative pathogens, such as Brachyspira pilosicoli, B. hyodysenteriae, Shigella sonnei, Vibrio cholera, V. parahaemolyticus, Campylobacter jejuni, Enterococcus faecium, Clostridium perfringens, Yersinia enterocolitica, Salmonella spp. and Bacillus cereus, as well as Listeria monocytogenes, Streptococcus pneumoniae, Enterococcus faecalis, Escherichia coli, Klebbsiella pneumoniae, Staphylococcus aureus, It is also active in vitro against Aspergillus niger and Fusarium chlamydosporum.

### BRIEF DESCRIPTION OF THE FIGURES

FIG. 1 is the 16S rRNA gene sequence of lactic acid bacteria strain D115 (SEQ. ID NO. 1).

FIG. 2 is the EF-Tu gene sequence of lactic acid bacteria strain D115 n(SEQ. ID NO. 2).

FIG. 3 is a graph of the effect of Lactobacillus johnsonii D115 on Brachyspira pilosicoli.

FIG. 4 is a graph of the effect of Lactobacillus johnsonii D115 on Brachyspira hyodysenteriae.

FIG. 5 is a graph of the effect of Lactobacillus johnsonii ATCC 11506 on Brachyspira hyodysenteriae.

FIG. 6 is a graph of the effect of Lactobacillus johnsonii ATCC 11506 on Brachyspira pilosicoli.

FIG. 7 is a graph of the effect of Lactobacillus johnsonii D115 on Salmonella typhimurium.

FIG. 8 is a graph of the effect of Lactobacillus johnsonii D115 on Salmonella enteritidis.

FIG. 9 is a graph of the effect of Lactobacillus johnsonii D115 on Clostridium perfringens.

FIG. 10 is the anti-fungal assay demonstrating the antifungal activity of (c and f) L. johnsonii D115 against A. niger compared to (a and d) the negative control and (b and e) L. johnsonii ATCC11506 for 14 and 21 days, respectively.

FIG. 11 is the well diffusion assay against Vibrio cholera. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIG. 12 is the well diffusion assay against Vibrio parahaemolyticus. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIG. 13 is the well diffusion assay against Shigella sonnei. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIG. 14 is the well diffusion assay against Campylobacter jejuni. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIG. 15 is the well diffusion assay against Streptococcus pneumoniae. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIG. 16 is the well diffusion assay against Enterococcus faecium. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism. The antibacterial effect of the D115 cell-free medium (a) can be seen clearly compared to the controls (b and c).

FIGS. 17A and 17B are charts of in vitro growth inhibition of Y enterocolitica by varying concentrations of reconstituted supernatant of L. johnsonii D115 (A) or L. johnsonii 15506 (B); growth was monitored at 37° C. by measuring the optical density at 600 nm in an automated Bioscreen C Analyser.

FIG. 18 is the well diffusion assay against Aspergillus niger. The antimicrobial effect of (a) 100 μl of L. johnsonii D115 cell-free culture medium, (b) MRS with 0.18% lactic acid and (c) L. johnsonii ATCC 11506 cell-free culture medium on the indicator organism.

### DESCRIPTION OF PREFERRED EMBODIMENTS

The present invention includes strains of Lactobacillus johnsonii that produce a heat-stable and pH tolerant metabolite(s) that has broad spectrum antimicrobial activity. The invention also includes such metabolite(s)s, the administration of the L. johnsonii strain as a probiotic which grows in the gastrointestinal tract of the animal or human to which it has been administered where it produces the metabolite(s), and to administration of the metabolite(s) for the prophylaxis of the effects of infections of Gram positive and Gram negative bacteria and fungi. The strain and the metabolite(s) are effective against Brachyspira pilosicoli, B. hyodysenteriae, Listeria monocytogenes, Shigella sonnei, Vibrio cholera, V parahaemolyticus, Campylobacter jejuni, Streptococcus pneumoniae, Enterococcus faecalis, Enterococcus faecium, Clostridium perfringens, Yersinia enterocolitica, Escherichia coli, Klebbsiella pneumoniae, Staphylococcus aureus, Salmonella spp., Bacillus cereus, Aspergillus niger and Fusarium chlamydosporum.

The metabolite(s) is heat stable, by which it is meant that the metabolite(s) has been subjected to heat treatment over time and found still to maintain its antimicrobial properties. The metabolite(s) has been found to maintain its activity when subjected to heat treatment throughout the range from ambient temperatures of about 20° C. up to and including 121° C. when such heat treatment has been applied over times of at least 15 min and more.

The metabolite(s) is also pH tolerant, by which it is meant that the metabolite(s) has been subjected to treatment under acidic conditions over time and found still to maintain its antimicrobial properties. The metabolite(s) has been found to maintain its activity when subjected to acidic conditions in throughout the range from neutral to and including pH1 when such acidic conditions have been applied over times of at least 30 min and more.

The present invention may be practiced by the oral administration of effective amounts of one or more bacterial strains such that a subject metabolite(s) is produced in vivo at levels that are antagonistic to the microbe of interest. Those skilled in the art will be able to determine the effective amount for particular applications through well-known methods. It is expected that an effective amount include doses in the range of approximately 10⁶ CFU to 10¹² CFU per day.

The present invention may also be practiced by the oral administration of an effective amount of a metabolite(s) to produce an antagonistic effect on the microbe of interest. Those skilled in the art will be able to determine the effective amount for particular applications through well-known methods.

The present invention also may be practiced by adding the effective amounts of one or more of the bacterial strains to a food or feed to prevent contamination by or inhibit the growth of a microbe of interest. Those skilled in the art will be able to determine the effective amount for particular applications through well-known methods.

### Example 1

### Materials and Methods

Culture conditions of lactic acid bacteria (LAB) strain D115. Lactic acid bacteria strain D115 was grown in deMan Rogosa Sharpe broth (MRS, pH 6.3) (Becton Dickinson and Company, USA) at 37° C. under anaerobic condition for 24 h. Overnight culture was streaked onto MRS agar and the arising pure colonies were sub-cultured in MRS broth using the same conditions as described. Cultures were kept in 20% glycerol at −80° C. for long-term storage.

Culture conditions of Brachyspira spp. Brachyspira hyodysenteriae ATCC 27164 and B. pilosicoli ATCC 51139 were grown in Brain Heart Infusion broth (Oxoid Ltd, Basingstoke, Hampshire, England) supplemented with 10% fetal calf serum (HyClone Laboratories Inc, USA), 0.05% L-cysteine (Sigma Chemical Co., Steinheim, Germany) and 0.2% glucose (Merck, Darmstadt, Germany), and incubated at 37° C. under strict anaerobic condition for 4-6 days. Cultures were kept in 40% glycerol for long term storage at −80° C.

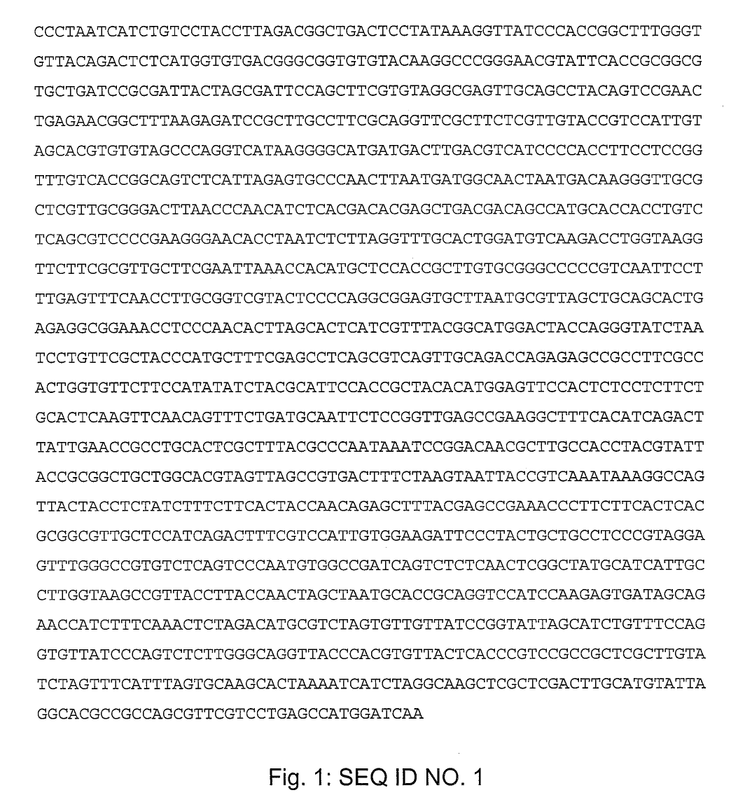

Bacterial identification by 16S rRNA sequencing. Isolated colonies of strain D115 were sent to Research Biolabs Technologies Pte Ltd, Singapore for sequencing work. The nearly full-length 16S rRNA was amplified by polymerase chain reaction (PCR) with forward primer 27F and reverse primer universal 1492R. Purified PCR products were sequenced using the ABI PRISM 3100 DNA sequencer and the ABI PRISM BigDye terminator cycle sequencing ready-reaction kit. Primers 27F, 530F, 926F, 519R, 907R and 1492R⁴⁴ were adopted to sequence both strands of the 16S rRNA gene. The sequences were finally assembled to produce the full-length sequence in FIG. 1 (SEQ ID NO. 1) and the full-length sequence was matched against NCBI Genbank database.

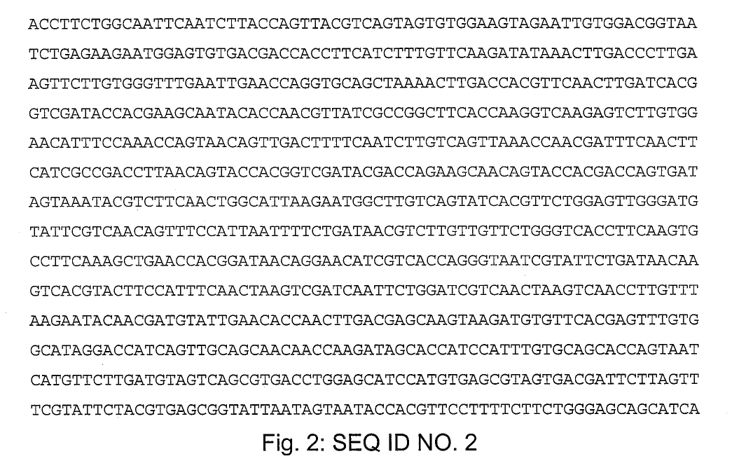

Bacterial identification by EF-Tu gene sequence. The approximately 900 bp tuf gene fragments were PCR-amplified using two oligonucleotide primers, namely TUF-1 (GATGCTGCTCCAGAAGA) and TUF-2 (ACCTTCTGGCAATTCAATC). The resultant PCR products were purified using Qiaquick PCR Purification Kit (Qiagen), and sequenced using an ABI PRISM 3100 DNA sequencer and ABI PRISM BigDye Terminator Cycle Sequencing ready-reaction kit. Two additional primers (TUF-f2-TGCTTCTGGTCGTATCGACCGT and TUF-f2-GGTCACCTTCAAGTGCCTTC) were designed and employed, together with the primers TUF-1 and TUF-2, for sequencing the PCR product in both directions. Finally, the sequences were assembled and the resultant sequence was compared with all other sequences available in the NCBI Genbank database. The EF-Tu gene sequence of lactic acid bacteria strain D115 is set out in FIG. 2. (SEQ ID NO. 2).

Antagonistic assay. Cultures of Lactobacillus johnsonii D115, Brachyspira hyodysenteriae and B. pilosicoli were centrifuged separately at 4200×g for 15 min before each was resuspended into phosphate-buffered saline (PBS). The pellet of L. johnsonii D115 was washed twice with PBS before resuspension. A 1-ml suspension of B. hyodysenteriae and B. pilosicoli was added into cells of L. johnsonii D115 to examine the antagonistic effect. Growth of B. hyodysenteriae and B. pilosicoli were also monitored in the absence of L. johnsonii D115.

In another set of sample flasks containing cells of L. johnsonii D115 and/or Brachypspira spp., 0.05% cysteine was added to determine the inhibitory effect of L. johnsonii D115 in the absence of hydrogen peroxide production. All samples flasks were incubated at 37° C. under anaerobic condition and shaking at 75 rpm. Samples were plated at 0, 2 and 4 h interval onto both MRS agar and Brain Heart Infusion agar supplemented with 5% defibrinated sheep blood (Oxoid, Basingstoke, Hampshire, England), 12.5 mg/l of rifampicin and 200 mg/l of spectinomycin. MRS agar and blood agar plates were incubated at 30° C. under 5% CO₂ and 37° C. under anaerobic condition, respectively.

Antagonistic assay. Overnight cultures of Lactobacillus johnsonii D115, C. perfringens, Salmonella enteritidis and S. typhimurium were centrifuged separately at 4200×g for 15 min. The pellet of L. johnsonii D115 was washed twice with PBS before re-suspending the pellet with 10 ml phosphate-buffered saline (PBS) to achieve a 10¹⁰ CFU/ml culture. The indicator organisms were re-suspended with PBS to achieve a 10⁷ CFU/ml culture. A 1-ml suspension of C. perfringens or Salmonella enteritidis or S. typhimurium was added individually to 9 ml of L. johnsonii D115 culture in 50 ml disposable BD Falcon® conical-bottom disposable plastic tubes. Individual tubes containing either only cultures of L. johnsonii D115 or C. perfringens or Salmonella enteritidis or S. typhimurium or cultures of L. johnsonii D115 and C. perfringens or Salmonella enteritidis or S. typhimurium, with 0.05% cysteine were included as controls. All cultures were incubated at 37° C. under aerobic condition, except C. perfringens which was in anaerobic condition, and shaking at 75 rpm. A 1 ml sample was removed at intervals of 0 and 4 h from each mixed culture and a 9-fold serial dilution was carried out before the samples were plated onto MRS agar and/or Perfringens agar and/or Tryptone Soy Agar supplemented with yeast extract (Oxoid, Basingstoke, Hampshire, England). Cultures were incubated at 37° C. under aerobic condition except for the Perfringens agar which was incubated at 37° C. under anaerobic condition.

Measurement of hydrogen peroxide production. Hydrogen peroxide production was determined using FOX-2 (ferrous-oxidation-xylenol 2) method at 0, 2 and 4 h interval during the antagonistic assay. Cell suspensions were centrifuged at 4200×g for 15 min before 190-μl volume of the supernatant was transferred to another microcentrifuge tube containing 10 μl of methanol for subsequent reaction with FOX-2 reagent. The reagent was prepared from 2,6-di-tert-butyl-4-methyphenol (>99%, Merck Schuchardt Germany), HPLC grade methanol (Merck, Germany), xylenol orange sodium salt (ACS reagent, Sigma Chemicals, St Louis, Mo.), ammonium ferrous sulfate (>99%, ACS reagent, Aldrich, USA), and sulfuric acid (95-97%, Merck, Darmstadt Germany). Three negative controls containing 1) bacterial supernatant and catalase, 2) PBS and methanol, and 3) PBS and catalase (1000 U/ml) were also incorporated. To each treatment, a 800-μl volume of the FOX-2 reagent was added, mixed well by agitation before centrifugation at 4200×g for 10 min. The optical density (OD) readings were recorded against a methanol blank using a spectrophotometer set at the wavelength of 560 nm and the concentration of hydrogen peroxide was determined from a standard curve.

Effect of hydrogen peroxide on Brachyspira spp. The average concentration of hydrogen peroxide produced by Lactobacillus johnsonii D115 at 2 and 4 h intervals in the antagonistic assay was determined. A 10 mM stock solution of hydrogen peroxide was prepared from 30% purity hydrogen peroxide (Merck, Germany) using PBS. The stock solution was added into culture of Brachyspira spp., previously resuspended in PBS, to achieve the pre-determined concentration of hydrogen peroxide as mentioned. Brachyspira spp. without addition of hydrogen peroxide was used as a control. Samples were incubated at 37° C. under anaerobic condition for 2 h. Plate count of Brachyspira spp. was performed using Brain Heart Infusion agar supplemented with 5% defibrinated sheep blood.

Extraction and separation of D115 active metabolite(s). Lactic acid bacteria strain D115 was grown in deMan Rogosa Sharpe broth (MRS, pH 6.3) (Becton Dickinson and Company, USA) at 37° C. under anaerobic condition for 24 h. The culture was centrifuged at 4200× g for 15 min. The supernatant was extracted three times using diethyl ether and the organic phase collected. The collected organic phase was evaporated off using a rota-evaporator and reconstituted using PBS. The extracted compounds were subjected to efficacy studies against Brachyspira hyodysenteriae, B. pilosicoli, C. perfringens, Salmonella enteritidis and S. typhimurium using well diffusion assay. The un-extracted culture broth was included as a control.

Effect of heat on active metabolite(s)(s). Lactic acid bacteria strain D115 was grown in deMan Rogosa Sharpe broth (MRS, pH 6.3) (Becton Dickinson and Company, USA) at 37° C. under 5% CO₂ for 48 h. The culture was centrifuged at 4200×g for 15 min. The supernatant was collected and subjected to moist heat at 121° C. and 100° C. for 15 min. The treated supernatant was cooled to room temperature and used in well diffusion assay against Brachyspira hyodysenteriae, B. pilosicoli, C. perfringens, Salmonella enteritidis and S. typhimurium. Heat-treated un-inoculated broth was included as a control.

Effect of pH on active metabolite(s)(s). Lactic acid bacteria strain D115 was grown in deMan Rogosa Sharpe broth (MRS, pH 6.3) (Becton Dickinson and Company, USA) at 37° C. under 5% CO₂ for 48 h. The culture was centrifuged at 4200×g for 15 min. The supernatant was collected and subjected to pH1 and 2 treatments at 40° C. for 30 min, respectively. The treated supernatant was used in well diffusion assay against Brachyspira hyodysenteriae, B. pilosicoli, C. perfringens, Salmonella enteritidis and S. typhimurium. pH-treated un-inoculated broth was included as a control.

Anti-fungal effect of D115. Cultures of Lactic acid bacteria strain D115 and Lactobacillus johnsonii ATCC 11506 strain were adjusted using phosphate-buffered saline (PBS, pH 7.4) to McFarland equivalent 0.5 unit. Each half of a yeast extract-supplemented Tryptone Soy Agar was inoculated with lactic acid bacteria strains, D115 and Lactobacillus johnsonii ATCC 11506, respectively, using the spread plate technique. The plates were incubated at 37° C. for 48 h. A point inoculation was made on the other half of the plates with either Aspergillus niger or Fusarium chlamydosporum. The plates were re-incubated at 30° C. for up to 7 (F. chlamydosporum) or 21 (A. niger) days.

### Results

Lactic acid bacteria strain D115 was isolated from the duodenum section of gastrointestinal tract of chicken. The preliminary bacterial identification using biochemical test (API 50 CHL) revealed the identity of the bacterium to be Lactobacillus fermentum. In the current study, the 16S rRNA sequencing results show that strain D115 belongs to the lactic acid bacteria group, however, to a different species, most probably Lactobacillus johnsonii (FIG. 1 and Table 1). Strain D115 exhibited highest gene sequence similarity with Lactobacillus johnsonii NCC533 at 100% and lowest similarity with Lactobacillus gasseri at 99.4% in the NCBI Genbank database (Table 1). The tuf gene sequencing results confirmed that strain D115 belongs to the lactic acid bacteria group and most probably Lactobacillus johnsonii (FIG. 2 and Table 2). Strain D115 exhibited highest tuf gene sequence similarity with Lactobacillus johnsonii NCC533 at 99.95% and lowest similarity with Lactobacillus jensenii ATCC 25258 at 91.20% in the NCBI Genbank database. Hence, the identity of strain D115 as Lactobacillus johnsonii was adopted in the subsequent work since 16S rRNA and the tuf gene sequencing have been accepted widely as a more reliable, simple and inexpensive way to identify and classify microbes.

TABLE 1
16S rRNA gene sequence identity search of lactic acid bacteria
strain D115 against known species in NCBI Genbank Database

| Bacteria strain | % identity |
|---|---|
| Lactobacillus johnsonii NCC 533 | 100 |
| Lactobacillus acidophilus johnsonii 16S ribosomal RNA | 99.87 |
| gene | |
| Lactobacillus gasseri strain ATCC 33323 16S ribosomal | 99.53 |
| RNA gene | |
| Lactobacillus gasseri strain KC5a 16S ribosomal RNA | 99.46 |
| gene | |
| Lactobacillus gasseri strain BLB1b 16S ribosomal RNA | 99.40 |
| gene | |

TABLE 2
tuf gene sequence identity search of lactic acid bacteria
strain D115 against known species in NCBI Genbank Database

| Bacteria strain | % identity |
|---|---|
| Lactobacillus johnsonii NCC 533 | 99.55 |
| Lactobacillus gasseri strain ATCC 33323 | 97.47 |
| Lactobacillus gasseri strain ATCC 19992 | 97.43 |
| Lactobacillus jensenii strain ATCC 25258 | 91.20 |

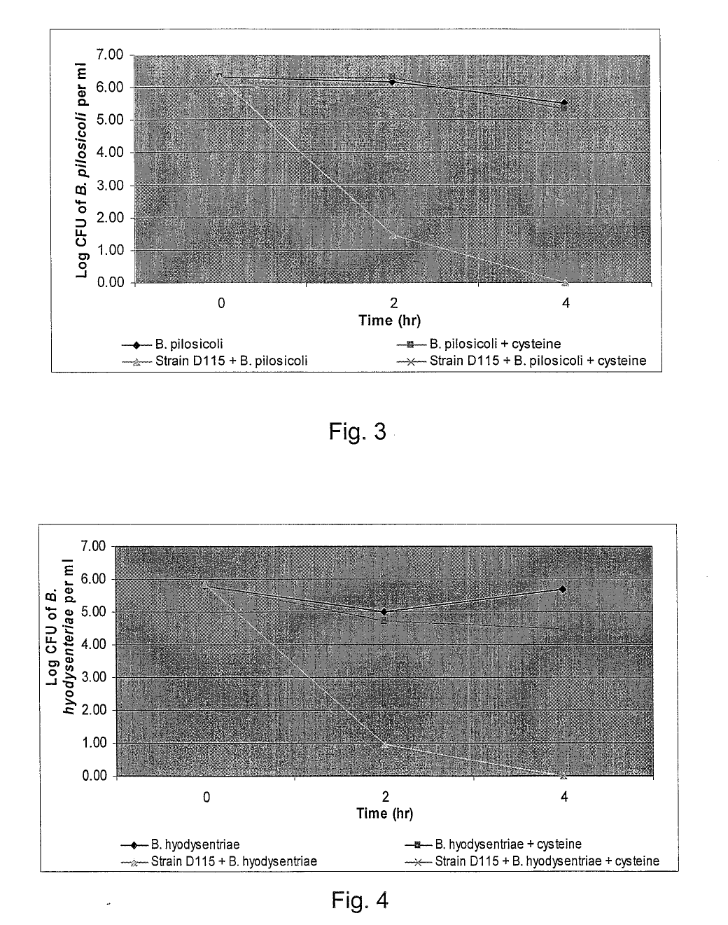

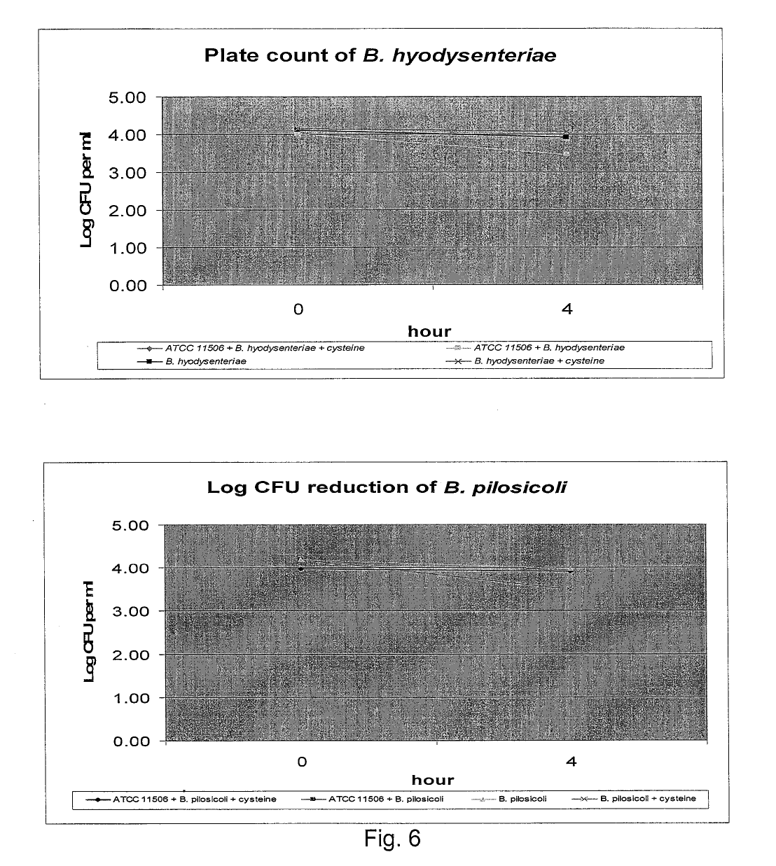

In the antagonistic assay against Brachyspira spp, production of hydrogen peroxide by L. johnsonii D115 was monitored at 0, 2 and 4 h intervals. Results obtained show the trend of increasing hydrogen peroxide production by strain D115 over the incubation period, with approximately 3,000 μM detected after 4 h incubation in both the antagonistic assays against B. pilosicoli and B. hyodysenteriae (Tables 3 and 4). However, when cells of L. johnsonii D115 were incubated with Brachyspira spp, an elevated amount of hydrogen peroxide (average of 3,400 μM) produced by the former bacterium was observed at 2 h interval, but then decreased in concentration in the subsequent incubation up to 4 h (Tables 3 and 4). No production of hydrogen peroxide was observed when strain D115 was incubated with the reducing agent, cysteine (Tables 3 and 4). Clear inhibitory effects of L. johnsonii D115 against both Brachyspira spp. were observed in this study (FIGS. 3 and 4).

TABLE 3
Production of hydrogen peroxide of
Lactobacilus johnsonii D115 in the presence of
Brachyspira pilosicoli

| | Production of hydrogen peroxide (μM)^{b} | | |
|---|---|---|---|
| Sample | 0 h | 2 h | 4 h |
| Strain D115^{a} | 616.2 | 1685.0 | 3016.7 |
| Strain D115 + B. pilosicoli | 778.8 | 3360.0 | 2933.3 |

^{a}Cells of L. johnsonii D115 was established at 10⁹ CFU per ml in all samples.
^{b}Production of hydrogen peroxide was not detected in samples containing cysteine.

TABLE 4
Production of hydrogen peroxide of Lactobacilus johnsonii D115 in
the presence of Brachyspira hyodysenteriae

| | Production of hydrogen peroxide (μM)^{b} | | |
|---|---|---|---|
| Sample | 0 h | 2 h | 4 h |
| Strain D115^{a} | 585.3 | 1310.0 | 3350.0 |
| Strain D115 + B. hyodysenteriae | 648.7 | 3510.0 | 2391.5 |

^{a}Cells of L. johnsonii D115 was established at 10⁹ CFU per ml in all samples.
^{b}Production of hydrogen peroxide was not detected in samples containing cysteine.

In the presence of hydrogen peroxide-producing strain D115, the bacterial counts of B. pilosicoli and B. hyodysenteriae were both reduced by 5 logs following 2 h incubation and complete inhibition was observed after 4 h incubation (FIGS. 3 and 4). No cells of Brachyspira spp. nor hemolytic activities were observed when the contents were plated on blood agar after 4 h (data not shown). Interestingly, these disease-causing spirochetes were also found to be susceptible to inhibition by L. johnsonii D115 even when hydrogen peroxide was removed by cysteine. Brachyspira pilosicoli and B. hyodysenteriae suffered 3 and 5 logs reduction in bacterial count respectively, in the absence of hydrogen peroxide (FIGS. 3 and 4). In this case, B. hyodysenteriae seems to be more susceptible to this additional antimicrobial compound produced by strain D115.

To further confirm and associate the killing effect of hydrogen peroxide on Brachyspira spp., we evaluated the survivability of these organisms in working solutions of hydrogen peroxide at the established concentration similar to that produced by L. johnsonii D115 in the antagonistic assays. Results show that hydrogen peroxide at approximately 3,000 μM reduced the count of B. hyodysenteriae and B. pilosicoli by 4 and 5 logs, respectively, similar to the inhibitory effect seen in the antagonistic assays (Tables 5 and 6). With regards to the antimicrobial compound in addition to hydrogen peroxide, we showed that the inhibitory effect of L. johnsonii D115 was not associated with the production of lactic acid, which can inhibit Brachyspira spp. Analytical testing using high performance liquid chromatography (HPLC) showed the absence or negligible traces of lactic acid in the culture suspensions containing both L. johnsonii D115 and Brachyspira spp. (data not shown).

TABLE 5
Effect of hydrogen peroxide on Brachyspira pilosicoli
over 2 h incubation

Plate count (CFU per ml)

| Sample | 0 hr | 2 hr |
|---|---|---|
| B. pilosicoli | 1.61 × 10⁶ | 1.08 × 10⁶ |
| B. pilosicoli + hydrogen | 6.00 × 10⁶ | 3.00 × 10¹ |
| peroxide^{a} | | |

^{a}Concentration of hydrogen peroxide was established at 3300 μM.

TABLE 6
Effect of hydrogen peroxide on Brachyspira hyodysenteriae
over 2 h incubation

Plate count (CFU per ml)

| Sample | 0 hr | 2 hr |
|---|---|---|
| B. hyodysenteriae | 5.9 × 10⁵ | 5.0 × 10⁵ |
| B. hyodysenteriae + hydrogen | 5.0 × 10⁵ | 9 |
| peroxide^{a} | | |

^{a}Concentration of hydrogen peroxide was established at 3100 μM.

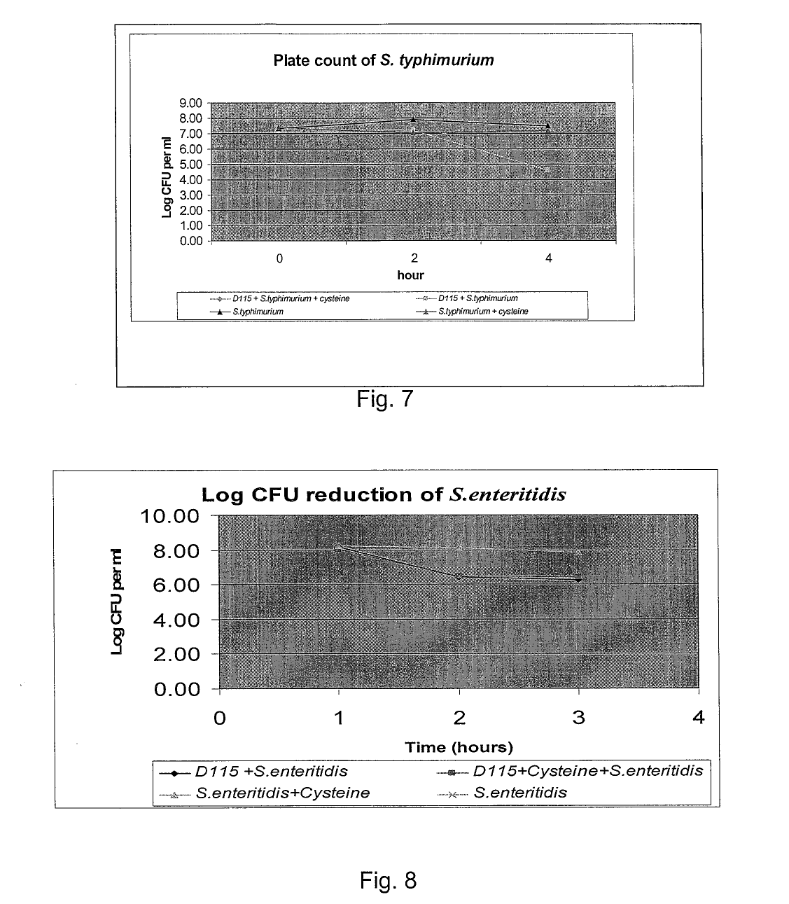

In the antagonistic assay conducted against Brachyspira pilosicoli and B. hyodysenteriae using Lactobacillus johnsonii ATCC 11506 strain, less than a one log reduction in the pathogenic bacteria was observed, as demonstrated in FIGS. 7 and 8. When hydrogen peroxide production was suppressed, almost no reduction of Brachyspira pilosicoli and B. hyodysenteriae was observed.

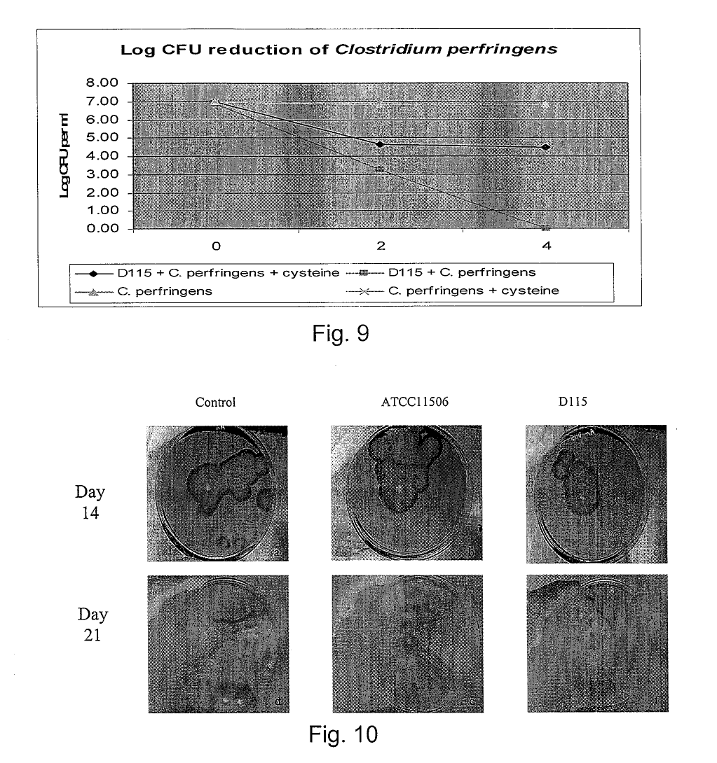

When strain D115 was tested against Salmonella typhimurium using the antagonistic assay, 2.5 logs reduction in the pathogenic bacterium was observed, as demonstrated in FIG. 9. When hydrogen peroxide production was suppressed with the reducing agent, a log reduction in Salmonella typhimurium was still observed, demonstrating that the inhibitory effect was due to the production of additional antimicrobial compound by strain D115.

When strain D115 was tested against Salmonella enteritidis using the antagonistic assay, 2 logs reduction in the pathogenic bacterium was observed, as demonstrated in FIG. 10. When hydrogen peroxide production was suppressed with the reducing agent, 2 logs reduction in Salmonella enteritidis was still observed, demonstrating that the inhibitory effect was due to the production of additional antimicrobial compound by strain D115.

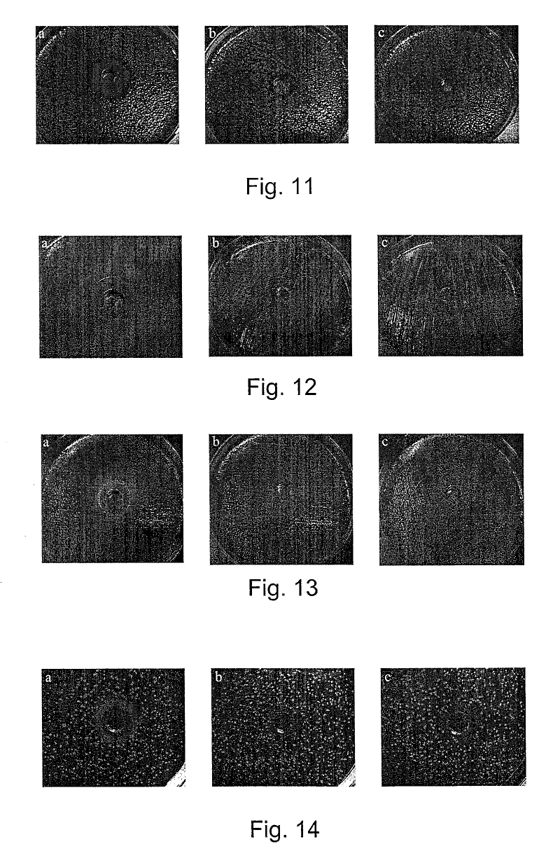

When strain D115 was tested against Clostridium perfringens using the antagonistic assay, 7 logs reduction in the pathogenic bacterium was observed, as demonstrated in FIG. 11. When hydrogen peroxide production was suppressed with the reducing agent, 2.5 logs reduction in Clostridium perfringens was still observed, demonstrating that the inhibitory effect was due to the production of additional antimicrobial compound by strain D115.

The 24-hr culture broth from strain D115 was subjected to 121° C. and 100° C. respectively for 15 min. The treated culture broth was tested for inhibitory effect against Brachyspira hyodysenteria, B. pilosicoli, Salmonella enteritidis, S. typhimurium and Clostridium perfringens in the well diffusion assay. As seen in Table 7, the heat-treated culture broth still demonstrated inhibitory effect against Brachyspira hyodysenteria, B. pilosicoli, Salmonella enteritidis, S. typhimurium and Clostridium perfringens. When the 24-hr culture broth from strain D115 was subjected to pH 1 and 2 treatments for 30 min at 40° C., the treated culture broth demonstrated inhibitory effect against Brachyspira hyodysenteria, B. pilosicoli, Salmonella enteritidis, S. typhimurium and Clostridium perfringens in the well diffusion assay, as seen in Table 8.

TABLE 7
Inhibitory Effect of Heat-Treated Culture Broth

| | B. pilosicoli | B. hyodysenteriae | S. enteritidis | S. typhimurium | C. perfringens |
|---|---|---|---|---|---|
| 100° C. | 5.0 | 5.0 | 2.5 | 1.5 | 4.0 |
| 121° C. | 5.0 | 5.0 | 2.5 | 1.5 | 4.0 |
| Untreated Broth | 5.0 | 5.0 | 3.5 | 2.5 | 4.0 |

TABLE 8
Inhibitory Effect of pH-Treated Culture Broth

| | B. pilosicoli | B. hyodysenteriae | S. enteritidis | S. typhimurium | C. perfringens |
|---|---|---|---|---|---|
| pH 1 | 5.0 | 5.0 | 2.0 | 1.0 | 4.0 |
| pH 2 | 5.0 | 5.0 | 2.0 | 1.0 | 4.0 |
| Untreated Broth | 5.0 | 5.0 | 3.0 | 2.0 | 4.0 |

The 24-hr D115 culture also demonstrated inhibition against Aspergillus niger and Fusarium chlamydosporum Compared to the plate with L. johnsonii ATCC 11506 in FIG. 10, the growth of the A. niger on the plate co-inoculated with D115 was suppressed. This could be attributed to the diffusion of anti-fungal compound(s) across the culture agar. On the other hand, the Aspergillus niger on the control plate with PBS demonstrated growth and spread of the fungus across the agar plate. L. johnsonii D115 also demonstrated inhibition against Fusarium chlamydosporum compared to L. johnsonii ATCC 11506 at day 7, as shown in Table 9.

TABLE 9

| | Average diameter of |
|---|---|
| Organism | F. chlamydosporum in mm |
| L. johnsonii D115 | 36.5 |
| L. johnsonii ATCC 11506 | 41.5 |

Comparatively, the Fusarium chlamydosporum that was co-incubated with D115 showed 13.7% suppression in size as compared to L. johnsonii ATCC 11506.

### Discussion

Strain D115 has been identified as Lactobacillus johnsonii using 16S rRNA sequencing in contrast to previous characterization as Lactobacillus fermentum using the API 50 CHL test. It is generally accepted that 16S rRNA sequencing has higher reliability compared to biochemical profiles. Sow et al, 2005 and Nigatu et al, 2000 demonstrated the insufficiency of API 50 CHL in the identification and the differentiation of Lactobacillus genus, and highlighted the need for genotyping techniques for more effective characterization^{50,40}. Evaluation of numerical analyses of RAPD and API 50 CH patterns to differentiate Lactobacillus plantarum, Lact. fermentum, Lact. rhamnosus, Lact. sake, Lact. parabuchneri, Lact. gallinarum, Lact. casei, Weiseella minor and related taxa isolated from kocho and tef. Journal of Applied Microbiology 89(6): 969-978). This is because phenotypic properties can be unstable at times and expression may be affected by evolution and environmental changes such as growth substrate, temperature and pH^{24,50}. Sequencing of the Elongation factor Tu (tuf) gene further confirmed the identity of the strain D115 to be under the genus of Lactobacillus species johnsonii. The tuf gene has been reported to be highly conserved throughout evolution and show functional constancy^{35,34}. Phylogenies based on protein sequences from elongation factor Tu has shown good agreement with the rRNA gene sequence data³⁵ and accurate for the identification of species within the Lactobacillus genus⁵³.

Lactobacillus johnsonii is a member of the acidophilus group for which probiotic roles have been well-reported⁴⁵. The bacterium was reclassified as a separate species from Lactobacillus acidophilus in 199217. Among the different strains of Lactobacillus johnsonii, strain NCC 533 (also known as strain La1)¹⁰ is the most well reported bacterium for its probiotic activities such as pathogen inhibition, epithelial cell attachment and immunomodulation^{12,20,39} The bacterium was found to be antagonistic towards Giardia intestinalis and protect against parasite-induced mucosal damage²⁰. Specifically in poultry, Lactobacillus johnsonii F19785 was reported to be able to suppress colonization of Clostridium perfringens through competitive exclusion³². These reports support the potential use of strain D115 as a probiotic against Brachyspira spp.

Our current study demonstrated two inhibitory actions of L. johnsonii D115 against Brachyspira spp. through the production of hydrogen peroxide and the presence of a second putative antimicrobial compound. Studies have shown that Lactobacillus spp. are capable of producing excessive hydrogen peroxide (H₂O₂) in an aerobic environment, thereby preventing the proliferation of other undesirable pathogenic bacteria that produce little or no H₂O₂-scavenging enzymes such as catalase^{5,15,31}. Lactic acid bacteria which are facultative anaerobes, convert molecular oxygen to hydrogen peroxide through their NADH oxidase system^{5,47}. Due to the absence of catalase, these bacteria depend solely on NADH peroxidase to keep hydrogen peroxide at sub-inhibitory concentration levels⁴⁷. In this case, the concentration of hydrogen peroxide produced by L. johnsonii D115 was shown to be inhibitory towards both B. hyodysenteriae and B. pilosicoli. The study by Philips et al (2003) also showed the use of hydrogen peroxide as a strong disinfectant to inactivate B. pilosicoli in the feces of chickens⁴³. The strong antimicrobial characteristic of hydrogen peroxide is due to its ability to cause breakage in DNA in bacteria^{4,51,}. Apart from hydrogen peroxide production, the second inhibitory action of L. johnsonii D115 is attributed to be due to the production of an antimicrobial compound and not lactic acid, as supported by the HPLC analysis. In fact, the production of other antimicrobial compounds besides organic acids by lactic acid bacteria is commonly reported^{7,13,33}. Specifically, Lactobacillus johnsonii La1 was also shown to produce bacteriocins which have a narrow inhibitory spectrum against Staphylococcus aureus, Listeria monocytogenes, S. typhimurium, Shigella flexneri, Klebsiella pneumoniae, Pseudomonas aeruginosa, and Enterobacter cloacae¹⁰.

L. johnsonii D115 was also demonstrated to be inhibitory against Salmonella spp. and C. perfringens using the antagonistic assay. When the reducing agent was added into the assay, inhibition can still be seen in all experiments against Salmonella spp. and C. perfringens, indicating the presence of antimicrobial compound(s) other than hydrogen peroxide. The antimicrobial compound(s) is more effective against Salmonella enteritidis compared to S. typhimurium.

Lactobacillus johnsonii D115 was also demonstrated to be inhibitory against Aspergillus niger. When the 24-hr old culture plate of strain D115 was co-incubated with A. niger, suppression of growth of A. niger was observed. This can be attributed to the anti-fungal compound(s) that has diffused across the culture agar. The culture plate containing co-incubation of L. johnsonii ATCC 11506 A. niger showed no suppression of the growth of the fungus. Control plate containing only PBS and the fungus also showed no suppressive effect on the fungus, with the fungal culture growing and spreading across the culture plate. In addition, it was observed that L. johnsonii D115 did not demonstrate inhibition against Penicillium chrysogenun. Currently, there are no reports of anti-Aspergillus and anti-Fusarium activity by L. johnsonii.

Overall, this study presents promising results in supporting the potential use of L. johnsonii D115 as an antimicrobial agent against Brachyspira spp. Most importantly, the idea of using lactic acid bacteria in the inhibition of these intestinal spirochetes is novel and provides a good alternative solution to the use of antibiotics in the treatment and prevention of swine dysentery and porcine intestinal spirochaetosis. In addition, the results also indicate the potential use of L. johnsonii D115 as an antimicrobial agent against Salmonella spp, C. perfringens, Aspergillus spp. and Fusarium spp. The idea of using lactic acid bacteria in the application on or in foods, feeds and animals for the prevention or inhibition of Salmonella spp, C. perfringens, Aspergillus spp. and Fusarium spp. contaminations is novel.

### Conclusion

This study demonstrated the potential use of Lactobacillus johnsonii D115 against both Brachyspira hyodysenteriae and B. pilosicoli. Lactobacillus johnsonii D115 was shown to inhibit both spirochetes with its production of hydrogen peroxide and another antimicrobial compound. The use of beneficial bacteria in the treatment and prevention of swine dysentery and porcine intestinal spirochaetosis is novel and may alleviate the current situation of increasing antibiotic resistance in pathogenic bacteria. Also, Lactobacillus johnsonii D115 was demonstrated to have inhibitory effect against Salmonella spp. and C. perfringens. Moreover, the antimicrobial compounds from strain D15 are heat tolerant up to 121° C. for 15 min and acid tolerant up to pH 1 for 30 min at 40° C. The results also indicate that Lactobacillus johnsonii D115 and its anti-microbial metabolite(s) is inhibitory against Aspergillus niger and Fusarium chlamydosporum.

### Example 2

### Material and Methods

Culture conditions of lactic acid bacteria (LAB) strain D115. Lactic acid bacteria strain D115 was grown in deMan Rogosa Sharpe broth (MRS, pH 6.3) (Becton Dickinson and Company, USA) at 37° C. under anaerobic condition for 24 h. Overnight culture was streaked onto MRS agar and the arising pure colonies were sub-cultured in MRS broth using the same conditions as described. Cultures were kept in 20% glycerol at −80° C. for long-term storage.

Culture conditions of indicator organisms. Campylobacter jejuni (ATCC 35918), Escherichia coli (ATCC 25922), Klebsiella pneumoniae (clinical isolate, National University Hospital, Singapore), Listeria monocytogenes (ATCC 7644), Shigella sonnei (clinical isolate, National University Hospital, Singapore), Vibrio cholera (clinical isolate, National University Hospital, Singapore), Vibrio parahaemolyticus (clinical isolate, National University Hospital, Singapore), Streptococcus pneumoniae (clinical isolate, National University Hospital, Singapore), Enterococcus faecalis (clinical isolate, National University Hospital, Singapore), Enterococcus faecium (clinical isolate, National University Hospital, Singapore), Aspergillus niger (ATCC 24126) and Fusarium chlamydosporum (ATCC 200468) were used as indicator organisms. Individual isolated colonies of Klebsiella pneumoniae, Escherichia coli and Aspergillus niger were streaked onto Nutrient agar respectively. Individual isolated colonies of Campylobacter jejuni, Shigella sonnei, Vibrio cholera and Vibrio parahaemolyticus were streaked onto Blood agar (Biomed Diagnostic, BBL) respectively, under microaerophilic condition using Campygen Pak (Oxoid). Campylobacter jejuni was incubated at 42° C. for 48 h. A single isolated colony of Streptococcus pneumoniae was streaked onto Blood agar. A single isolated colony of Listeria monocytogenes was streaked onto Brain Heart Infusion agar (Oxoid). Individual isolated colonies of Enterococcus faecalis and Enterococcus faecium were streaked onto MRS respectively. All cultures were incubated at 37° C. for 24 h unless stated otherwise.

Well diffusion assay. Isolated colony of each indicator organisms was re-suspended in phosphate-buffered saline and adjusted to a McFarland no. 0.5 standard except for A. niger, which was adjusted to a McFarland no. 0.1 standard. The A. niger inoculum was subjected to enumeration with a hemocytometer to confirm an initial density of 10⁶ conidia/ml. A sterile swab was dipped into each individual sample preparation and spread onto their respective growth agar uniformly. Wells were made into the agars using a sterile cork borer (number 5). A 100 μl of the L. johnsonii D115 cell-free medium was added into each well. The L. johnsonii ATCC 11506 cell-free medium and the respective uninoculated growth media were included as controls.

Microtiter plate growth assay. To quantitate the efficacy of L. johnsonii D115 supernatant as an antimicrobial against several bacteria, an automated growth inhibition assay in a microtiter plate was performed using a Bioscreen C Analyser (Thermo Labsystems, Thermo Electron Oy, Finland). In this method, turbidity at a wavelength of 600 nm was measured periodically and recorded as an indication of microbial growth. One hundred twenty five μL of the L. johnsonii D115 supernatant was combined with 125 μL of the test microorganism (M.O.) into individual wells of a Honeycomb microtiter plate (Thermo Electron), resulting in a total volume of 250 μL per well. Negative controls consisted of 125 μL of test organism and 125 μL of sterile distilled water. Blanks consisted of 125 μL of culture medium (no M.O.) and 125 μL of sterile distilled water. The incubation temperature was set to 37° C. for the bacteria, with a measurement interval of 10 min, after shaking. Data was collected over a 20-48 h period of time, depending on the growth rate of the microorganism.

Disk diffusion assay—microaerobic and anaerobic bacteria. Cells were grown on tryptone soy agar (TSA) plates supplemented with sheep blood under microaerobic or anaerobic conditions at 37° C. for 48 h. Cells were collected from each plate and resuspended in 3 mL of saline (1% peptone, 8.5% NaCl, 0.05% Triton-X-100). The OD₆₂₅ of each suspension was measured and adjusted to 0.08, as described above. One hundred μL of each standardized culture was plated on a TSA plate supplemented with sheep blood and left to dry. Five sterile paper disks were placed on the plate. Ten μL of the reconstituted L. johnsonii D115 supernatant or the L. johnsonii ATCC 11506 supernatant (negative control) was spotted on the disks. Plates were kept at 4° C. for four hours, in the appropriate atmospheric condition (microaerobic or anaerobic), prior to incubation overnight at 37° C.

### Results

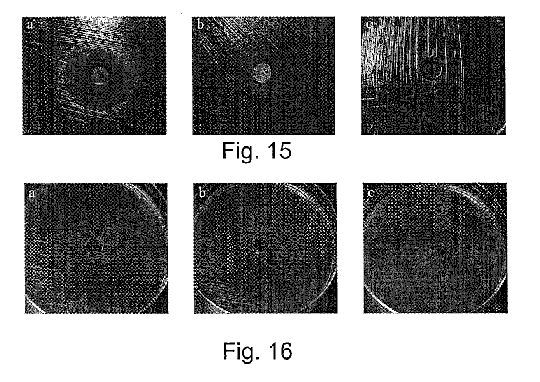

The viability of a variety of gram-positive and gram-negative microorganisms in the presence of L. johnsonii D115 cell-free medium was examined, in vitro, using the well diffusion assay. All bacteria tested were found to be sensitive to the antimicrobial compound(s) produced by L. johnsonii D115, with varying degrees of sensitivity (FIG. 11-16). As the average lactic acid concentration was found to be 1800 ppm or 0.18%, MRS containing 0.18% lactic acid was included as a negative control.

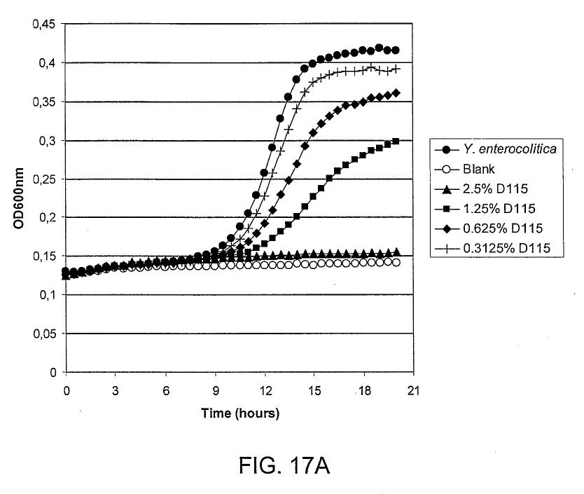

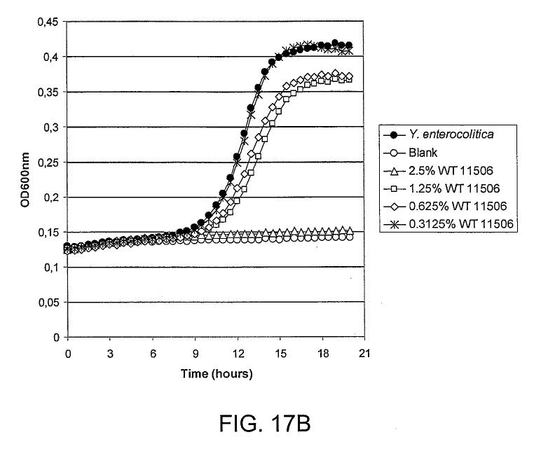

Microtiter plate growth assay. The OD of each well of the microtiter plate was measured every 10 min for 20-48 h (depending on the growth rate of the microorganism). A delay in the increase in OD₆₀₀ indicated an inhibition of cell growth by the antimicrobial solution. According to the results of the microtiter plate growth assay, the growth curves obtained indicate that in presence of the L. johnsonii D115 supernatant growth of Y enterocolitica was reduced compared to growth in presence of the L. johnsonii 11506 supernatant (FIGS. 17A and B)

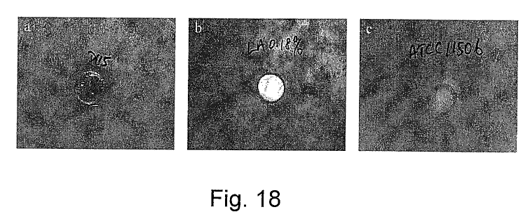

Antifungal screening using well diffusion assay confirmed that L. johnsonii D115 is active against the growth of common feed spoilage fungi such as Aspergillus niger (FIG. 18), as previously observed in example 1.

Disk diffusion assay. The diameter of the growth inhibition zone was measured using a ruler. When no inhibition was observed, the diameter was 6 mm, i.e. the diameter of the paper disk. Results are presented in Tables 10 and 11.

TABLE 10
Results of the disk diffusion assay screening
D115 supernatant against various bacteria

| | | Positive | Negative | Inhibition |
|---|---|---|---|---|
| | Source | Control¹ | control² | Increase |
| Microorganism | ID# | Avg. | Avg. | mm |
| B. cereus | ATCC 11778 | 28.0 | 25.8 | 2.3 |
| E. coli WT K-12 | ATCC 25404 | 18.9 | 16.0 | 2.9 |
| Y. enterocolitica | ATCC 9610 | 11.5 | 7.0 | 4.5 |
| S. montevideo | ATCC 8387 | 17.7 | 10.3 | 7.4 |
| S. senftenberg | ATCC 43845 | 20.5 | 16.1 | 4.4 |

¹L. johnsonii, Strain D115
²L. johnsonii ATCC 11506

### Discussion

Using the well diffusion assay method, several bacteria were shown to be susceptible to the putative antimicrobials contained in the L. johnsonii D115 supernatant, including Shigella sonnei, Vibrio cholera, V parahaemolyticus, Campylobacter jejuni, Streptococcus pneumoniae and Enterococcus faecium.

Using the well diffusion assay method, the anti-fungal activity of L. johnsonii D115 supernatant was further demonstrated against A. niger (FIG. 18). There was slight anti-fungal activity seen from the L. johnsonii ATCC 11506 cell-free culture medium but the inhibition zone detected using the L. johnsonii D115 cell-free culture medium demonstrated clear and defined inhibition of the fungus. The results demonstrated that the L. johnsonii D115 supernatant had a growth inhibitory activity against these microorganisms compared to the L. johnsonii 11506 supernatant. The effect was not due to the lactic acid production, common to lactic acid bacteria; this antimicrobial effect was due to the production of a secondary metabolite(s).

Using the disk diffusion assay method several bacteria were shown to be susceptible to metabolite(s) contained in the L. johnsonii D115 supernatant, including Salmonella montevideo, S. senftenberg, E. coli, Bacillus cereus and Y. enterocolitica.

Overall, L. johnsonii D115 has shown broad-spectrum anti-bacterial and anti-fungal activity, as summarized in Table 11 below.

TABLE 11
Summary of the results of the antimicrobial
activity of L. johnsonii D115

| | Zone of inhibition |
|---|---|
| Organism | (mm radius) |
| Brachyspira pilosicoli (ATCC51139) | 4.8 |
| Brachyspira hyodysenteriae (ATCC27164) | 5.0 |
| Escherichia coli (ATCC25922) | 4.2 |
| Salmonella enteritidis (ATCC13076) | 5.0 |
| Salmonella typhimurium (NUH clinical isolate) | 5.8 |
| Clostridium perfringens (NUH clinical isolate) | 5.2 |
| Klebsiella pneumoniae (NUH clinical isolate) | 5.4 |
| Campylobacter jejuni (ATCC 35918) | 4.4 |
| Listeria monocytogenes (NUH clinical isolate) | 4.6 |
| Shigella sonnei (NUH clinical isolate) | 4.5 |
| Vibrio cholera (NUH clinical isolate) | 4.9 |
| Vibro parahaemolyticus (NUH clinical isolate) | 5.1 |
| Streptococcus pneumoniae (NUH clinical isolate) | 5.8 |
| Enterococcus faecium (NUS-NR 10/10 IL8) | 10.5 |
| Enterococcus faecalis (NUS-EL 7/10 P4) | 6.3 |
| Aspergillus niger (ATCC 24126) | 3.7 |
| Fusarium chlamydosporum (ATCC200468) | 3.4 |
| E. coli (ATCC 25922 - LMG 8223) | 2.4 |
| Salmonella typhimurium (ATCC 700408) | 1.1 |
| Shigella sonnei (ATCC 25931 - LMG 10473) | 1.6 |
| Yersinia enterocolitica (ATCC 9610 - LMG 7899^{T}) | 2.3 |
| Bacillus cereus (ATCC 11778) | 1.2 |
| Escherichia coli WT K-12 (ATCC 25404) | 1.5 |
| Salmonella Montevideo (ATCC 8387) | 3.7 |
| Salmonella senftenberg (ATCC 43845) | 2.2 |

The Lactobacillus johnsonii isolate D115 was deposited under the terms of the Budapest Treaty at the American Type Culture Collection (ATCC) 10801 University Boulevard, Manassas, Va. 20110-2209 on Mar. 7, 2008, as PTA-9079.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### Conclusion

This study demonstrated the broad-spectrum anti-bacterial and anti-fungal activity of Lactobacillus johnsonii D115. In addition to what has been reported of other L. johnsonii strain, such as L. johnsonii La1, with inhibitory effect against Staphylococcus aureus, Listeria monocytogenes, S. enteritidis, S. typhimurium, Klebsiella pneumoniae, E. facalis, E. coli, the L. johnsonii D115 strain was found to be inhibitory against Shigella sonnei, Vibrio cholera, V. parahaemolyticus, Campylobacter jejuni, Streptococcus pneumoniae, Enterococcus faecium, Yersinia enterocolitica, Bacillus cereus, Aspergillus niger and Fusarium chlamydosporum. This indicates the potential use of Lactobacillus johnsonii D115 as a probiotic, as a prophylactic agent or as a surface treatment of materials against human and animal pathogens such as Shigella sonnet, Vibrio cholera, V parahaemolyticus, Campylobacter jejuni, Streptococcus pneumoniae, Enterococcus faecium, Yersinia enterocolitica, Bacillus cereus, S. Montevideo and S. senftenberg and the fungi Aspergillus niger and Fusarium chlamydosporum.

### REFERENCE

1. Aarestrup F. M. 1995. Occurrence of glycopeptide resistance among Enterococcus faecium isolates from conventional and ecological poultry farms. Microb Drug Resist. 1: 255-257.
2. Abarca M. L, M. R. Bragulat, G. Castella and F. J. CabanesS. 1994. Ochratoxin A Production by Strains of Aspergillus niger var. niger. Applied and environmental Microbiology. 60: 2650-2652.
3. Amann, R., Ludwig, W. & Schleifer, K. H. 1988. Subunit of ATP-synthase: a useful marker for studying the phylogenetic relationship of eubacteria. J Gen Microbiol. 134: 2815-2821.
4. Ananthaswamy H. N and Eisenstark A (1977) Repair of hydrogen peroxide-induced single-strand breaks in Escherichia coli deoxyribonucleic acid. Journal of Bacteriology 130(1): 187-191.
5. Angeles-Lopez M., E. G. Ramos E. G. C. and C. A. Santiago (2001) Hydrogen peroxide production and resistance to nonoxinol-9 in Lactobacillus spp. isolated from vagina of reproductive age women. Revista Latinoamericana de Microbiologia 43(4): 171-176.
6. Angeles-Lopez, et al., 2001; Sanders J. W., G. Venema and J. Kok (1999) Environmental stress response in Lactococcus lactis, FEMS Microbiology Reviews 23: 482-501.
7. Avonts L. and L. De Vuyst (2001) Antimicrobial potential of probiotic lactic acid bacteria. Meded Rijksuniv Gent Fak Landbouwkd Toegep Biol Wet. 66(3b): 543-550.
8. Barcellos D. E., M. R. Matliesen, M. D. Uzeka, Kader and G. E. Duhamel (2000) Prevalence of Brachyspira species isolated from diarrhoeic pigs in Brazil. The Veterinary Record 146(4): 398-403.
9. Bates J., Jordens J. Z., Griffiths D. T. 1994. Farm animals as a putative reservoir for vancomycin-resistant enterococcal infection in man. J Antimicrob Chemother. 34: 507-514.
10. Bernet-Camard M., V. Lievin, D. Brassart, J. Neeser, A. L. Servin and S. Hudault. 1997. The Human Lactobacillus acidophilus Strain LA1 Secretes a Nonbacteriocin Antibacterial Substance(s) Active In Vitro and In Vivo. Applied and Environmental Microbiology. 63(7): 2747-2753.
11. Black J. G. 1993. Microbiology: Principle and Applications. 2^{nd} edition. Prentice Hall. p 617.
12. Cruchet S., M. C. Obregon, G. Salazar, E. Diaz and M. Gotteland (2003) Effect of the ingestion of a dietary product containing Lactobacillus johnsonii La1 on Helicobacter pylori colonization in children. Nutrition 19(9): 716-721.
13. De Souza E. L., C. A. Da Silva and C. P. De Sousa (2005) Bacteriocins: Molecules of fundamental impact on the microbial ecology and potencial food biopreservatives. Brazilian Archives of Biology and Technology 48(4): 559-566.
14. Duhamel G. E., J. M. Kinyon, M. R. Mathiesen, D. P. Murphy and D. Walter (1998) In vitro activity of four antimicrobials agents against North American isolates of porcine Serpulina pilosicoli. Journal of Veterinary Diagnostic Investigation. 10: 350-356; Notvatna et al., 2002.
15. Eschenbacli D. A., P. R. Davick, B. L. Williams, S. J. Klebanoff, K. Y. Smith, C. M. Critchlow and K. K. Holmes (1989) Prevalence of hydrogen peroxide-producing Lactobacillus species in normal women and women with bacterial vaginosis. Journal of Clinical Microbiology 27(2): 251-256.
16. Fossi M., T. Saranpaa and E. Rautiainen. 1999. In vitro sensitivity of the swine Brachyspira species to tiamulin in Finland 1995-1997. Acta Veterinaria Scandinavica 40: 355-358.
17. Fujisawa T., Y. Benno, T. Yaeshima and T. Mitsuoka (1992) Taxonomic study of the Lactobacillus acidophilus group, with recognition of Lactobacillus fallinarum sp. nov. and Lactobacillus johnsonii sp. nov. and synonymy of Lactobacillus acidophilus group A3 (Johnson et al. 1980) with the type strain of Lactobacillus amylovorus (Nakamura 1981). International Journal of Systematic Bacteriology 42: 487-491.
18. Henrichsen, J. 1995. Six newly recognized types of Streptococcus pneumoniae. J. Clin. Microbiol. 33:2759-2762.
19. Hommez J., L. A. Devriese, F. Castryck, C. Miry., A. Lein and F. Haesebrouck (1998) Susceptibility of different Serpuilina species in pigs to antimicrobial agents. Vlaams Diergeneeskundig Tijdschrift 67: 32-35.
20. Humen M. A., G. L. D. Antoni, J. Benyacoub, M. E. Coastas et al. (2005) Lactobacillus johnsonii La1 antagonizes Giardia intestinales in vivo. Infection and Immunity 73(2): 1265-1269.
21. Humphrey T., O'Brien S, and Madsen M. 2007. Campylobacters as zoonotic pathogens: A food production perspective. International Journal of Food Microbiology 117: 237-257.
22. Hyatt D. R., A. A. H. M. Huurne, B. A. M. Van Der Zeijst and L. A. Joens (1994) Reduced virulence of Serpulina hyodysenteriae hemolysin-negative mutants in pigs and their potential to protect pigs against challenge with a virulent strain. Infection and Immunity 62(6): 2244-2248.
23. Jacobson M., C. Fellstrom, R. Lindberg, P. Wallgren and M. Jensen-Waern (2004) Experimental swine dysentery: comparison between infection models. Journal of Medical Microbiology 53: 273-280.
24. Janda, J. M. and S. L. Abbott (2002) Guest Commentary: Bacterial identification for publication: When is enough enough?. Journal of Clinical Microbiology 40(6): 1887-1891.
25. Jensen T. K., K. Moller, M. Boye, T. D. Leser and S. E. Jorsal (2000) Scanning electron microscopy and fluororescent in situ hybridization of experimental Brachyspira (Serpulina) pilosicoli infection in growing pigs. Vet. Pathol. 37: 22-32.
26. Jorgsten F., Bailey R., Williams S., Henderson P., Wareing D. R., Bolton F. J., Frost J. A., Ward L. and Humphrey T. J. 2002. Prevalence and numbers of Salmonella and Campylobacter spp. on raw, whole chickens in relation to sampling methods. International Journal of Food Microbiology 76:151-164.
27. Karlsson M., S. L. Oxberry and D. J. Hampson (2002) Antimicrobial susceptibility testing of Australian isolates of Brachyspira hyodysenteriae using a new broth dilution method. Veterinary Microbiology 84: 123-133.
28. Kennedy M. J. And R. J. Yancey Jr. (1996) Motility and chemotaxis in Serpulina hyodysenteriae. Veterinary Microbiology 49: 21-30.
29. Kennedy M. J., Rosnick D. K., Ulrich, R. G. and Yancey R. J. (1988) Association of Treponema hyodysenteriae with porcine intestinal mucosa. Journal of General Microbiology 134: 1565-1576.
30. Klare I., Heier H., Claus H., Reissbrodt R., Witte W. 1995. vanA-Mediated high-level glycopeptide resistance in Enterococcus faecium from animal husbandry. FEMS Microbiol Lett. 25: 165-171.
31. Klebanoff S. J. S. L. Hiller, D. A. Eschenbach and A. M. Waltersdorph (1991) Control of the microbial flora of the vagina by H2O2-generating lactobacilli. Journal of Infectious Diseases 164(1): 94-100.
32. La Ragione, R. M., A, Narbad, M. J. Gasson and M. J. Woodward (2004) In vivo characterization of Lactobacillus johnsonii FI9785 for use as a defined competitive exclusion agent against bacterial pathogens in poultry. Letters in Applied Microbiology 38: 197-205.
33. Lowe D. P. and E. K. Arendt (2004). The use and effects of lactic acid bacteria in malting and brewing with their relationships to antifungal activity, mycotoxins and gushing. A Review. J. Inst. Brew. 110(3): 163-180.
34. Ludwig, W., Neumaier, J., Klugbauer, N. & 9 other authors 1993. Phylogenetic relationships of bacteria based on comparative sequence analysis of elongation factor Tu and ATP-synthase beta-subunit genes. Antonie van Leeuwenhoek. 64: 285-305.
35. Ludwig, W., Weizenegger, M., Betzl, D., Leidel, E., Lenz, T., Ludvigsen, A., Mollenhoff, D., Wenzig, P. & Schleifer, K. H. 1990. Complete nucleotide sequences of seven eubacterial genes coding for the elongation factor Tu: functional, structural and phylogenetic evaluations. Arch Microbiol. 153: 241-247.
36. Maciorowski K. G, P. Herrera, M. M. Kundinger and S. C. Ricke. 2006. Animal Feed Production and Contamination by Foodborne Salmonella. Journal of Consumer Protection and Food Safety. 1: 197-209.
37. Muir S., M. B. H. Koopman, S. J. Libby, L. A. Joens, F. Heffron and J. G. Kusters (1992) Cloning and expression of a Serpula (Treponema) hyodysenteriae hemolysin gene. Infection and Immunity 60(2): 529-535.
38. Nachamkin 1.2002. Chronic effects of Campylobacter infection. Microbes and Infection 4: 399-403.
39. Neeser J. R., Granato D., M. Rouvet., A. Servin., S. Teneberg and K. A. Karlsson (2000) Lactobacillus johnsonii La1 shares carbohydrate-binding specificities with several enteropathogenic bacteria. Glycobiology 10(11): 1193-1199.
40. Nigatu (2000). Evaluation of numerical analyses of RAPD and API 50 CH patterns to differentiate Lactobacillus plantarum, Lact. fermentum, Lact. rhamnosus, Lact. sake, Lact. parabuchneri, Lact. gallinarum, Lact. casei, Weiseella minor and related taxa isolated from kocho and tef. Journal of Applied Microbiology 89(6): 969-978.
41. Novotna M. and O, Skardova. 2002. Brachyspira hyodysenteriae: detection, identification and antibiotic susceptibility. Veterinary Medicine 47(4): 104-109.
42. Oxberry S. L. and D. J. Hampson (2003) Epidemiological studies of Brachyspira pilosicoli in two Australian piggeries. Veterinary Microbiology 93: 109-120.
43. Philips N. D., T. La and D. J. Hampson. 2003. Survival of intestinal spirochaete strains from chickens in the presence of disinfectants and in faeces held at different temperatures. Avian Pathology 32(6): 639-643.
44. Placinta C. M, J. P. F. D'Mello, and A. M. C. MacDonald. (1999). A review of worldwide contamination of cereal grains and animal feeds with Fusarium mycotoxins. Anim. Feed Sci. Technol. 78:21-37.
45. Pridmore R. D., B. Berger., F. Desiere, D. Vilanova, C. Barretto et al. (2004) The genome sequence of the probiotic intestinal bacterium Lactobacillus johnsonii NCC 533. Proc. Natl. Acad. Sci. 101(8): 2512-2517.
46. Reid G. (1999) The scientific basis for probiotic strains of Lactobacillus. Applied and Environmental Microbiology 65(9): 3763-3766.
47. Sanders J. W., G. Venema and J. Kok 1999. Environmental stress response in Lactococcus lactis, FEMS Microbiology Reviews 23: 482-501
48. Skirrow S. (2002) Swine dysentery. Department of Agriculture, Government of Western Australia, WA 6983.
49. Smith, S. C., T. Muir, M. Holmes and P. J. Coloe. 1991. In vitro antimicrobial susceptibility of Australian isolates of Treponema hyodysenteriae. Australian Veterinary Journal 68: 408-409.
50. Sow N. D. M., R. D. Dauphin, D. Roblain, A. T. Guiro and P. Thonart (2005) Polyphasic identification of a new thermotolerant species of lactic acid bacteria isolated from chicken faeces. African Journal of Biotechnology 4(5): 409-421
51. Steiner B. M., H. W. G. H. W. Wong, P. Suprave and S. Graves (1984) Oxygen toxicity in Treponema pallidum: deoxyribonucleic acid single-stranded breakage induced by low doses of hydrogen peroxide. Canadian Journal of Microbiology. 30: 1467-1476.
52. Thomson J. R., W. J. Smith, B. P. Murray, S. McOrist (1997) Pathogenicity of three strains of Serpulina pilosicoli in pigs with a naturally acquired intestinal flora. Infection and Immunity 65(9): 3693-3700.
53. Ventura M., C. Canchaya, V. Meylan, T. R. Klaenhammer and R. Zink. 2003. Analysis, Characterization, and Loci of the tuf Genes in Lactobacillus and Bifidobacterium Species and Their Direct Application for Species Identification. App. Env. Microbio. 69(11): 6908-6922.

We claim:

1. An isolated bacterium of Lactobacillus johnsonii strain identified as D115 and deposited with the ATCC under deposit number PTA-9079.

2. The isolated bacterial strain as defined in claim 1, further comprising the sequence of SEQ ID NO. 1.

3. The isolated bacterial strain as defined in claim 2, wherein the strain has at least 90% homology sequence of SEQ ID NO. 1.

4. The isolated bacterial strain as defined in claim 1, further comprising the sequence of SEQ ID NO. 2.

5. The isolated bacterial strain as defined in claim 4, wherein the strain has at least 90% homology to the tuf gene sequence of SEQ ID NO. 2

6. A composition, comprising:
(a) bacterial cells of the genus Lactobacillus species johnsonii strain D115 that produce an anti-microbial metabolite that is heat stable at temperature up to 121° C. for at least 15 minutes and is acid-tolerant in the range from neutral to pH 1 for at least 30 minutes; and
(b) a physiologically acceptable carrier for the bacterial cells and metabolite, suitable for oral administration.

7. The composition according to claim 6, wherein the metabolite has anti-microbial activity against human and animal pathogens.

8. The composition according to claim 7, wherein the human and animal pathogens are selected from the group consisting of Brachyspira spp., Shigella spp., Vibrio spp., Campylobacter spp., Streptococcus spp., Enterococcus spp., Listeria spp., Clostridium spp., Klebbsiella spp., Staphylococcus spp., Salmonella spp., Yersinia enterocolitica, Escherichia coli, Bacillus cereus, Aspergillus niger and Fusarium chlamydosporum.

9. A method for the prophylaxis of the effects of an infection of microbes selected from the group consisting of Brachyspira spp., Shigella spp., Vibrio spp., Campylobacter spp., Streptococcus spp., Enterococcus spp., Listeria spp., Clostridium spp., Klebbsiella spp., Staphylococcus spp., Salmonella spp., Yersinia enterocolitica, Escherichia coli, Bacillus cereus, Aspergillus niger and Fusarium chlamydosporum, comprising the step of administering an effective amount of the composition of or metabolite(s) of the strain of claim 6.

10. A method for the prophylaxis of the effects of an infection of microbes selected from the group consisting Brachyspira spp., Shigella spp., Vibrio spp., Campylobacter spp., Streptococcus spp., Enterococcus spp., Listeria spp., Clostridium spp., Klebbsiella spp., Staphylococcus spp., Salmonella spp., Yersinia enterocolitica, Escherichia coli, Bacillus cereus, Aspergillus niger and Fusarium chlamydosporum, comprising the step of administering an effective amount of a strain of claim 6.

11. A method of treating a material to inhibit contamination by microbes selected from the group consisting Brachyspira spp., Shigella spp., Vibrio spp., Campylobacter spp., Streptococcus spp., Enterococcus spp., Listeria spp., Clostridium spp., Klebbsiella spp., Staphylococcus spp., Salmonella spp., Yersinia enterocolitica, Escherichia coli, Bacillus cereus, Aspergillus niger and Fusarium chlamydosporum, comprising the step of administering an effective amount of the strain of claim 6 to the material.

12. A method of treating a material to inhibit growth of microbes selected from the group consisting Brachyspira spp., Shigella spp., Vibrio spp., Campylobacter spp., Streptococcus spp., Enterococcus spp., Listeria spp., Clostridium spp., Klebbsiella spp., Staphylococcus spp., Salmonella spp., Yersinia enterocolitica, Escherichia coli, Bacillus cereus, Aspergillus niger and Fusarium chlamydosporum, comprising the step of administering an effective amount of the strain of claim 6 to the material.


SEQUENCE LISTING (2 sequences)

SEQ ID NO: 1 - DNA, 1509 bp, Lactobacillus johnsonii
ccctaatcatctgtcctaccttagacggctgactcctataaaggttatcccaccggctttgggtgttacagactctcatggtgtgacgggcggtgtgtacaaggcccgggaacgtattcaccgcggcgtgctgatccgcgattactagcgattccagcttcgtgtaggcgagttgcagcctacagtccgaactgagaacggctttaagagatccgcttgccttcgcaggttcgcttctcgttgtaccgtccattgtagcacgtgtgtagcccaggtcataaggggcatgatgacttgacgtcatccccaccttcctccggtttgtcaccggcagtctcattagagtgcccaacttaatgatggcaactaatgacaagggttgcgctcgttgcgggacttaacccaacatctcacgacacgagctgacgacagccatgcaccacctgtctcagcgtccccgaagggaacacctaatctcttaggtttgcactggatgtcaagacctggtaaggttcttcgcgttgcttcgaattaaaccacatgctccaccgcttgtgcgggcccccgtcaattcctttgagtttcaaccttgcggtcgtactccccaggcggagtgcttaatgcgttagctgcagcactgagaggcggaaacctcccaacacttagcactcatcgtttacggcatggactaccagggtatctaatcctgttcgctacccatgctttcgagcctcagcgtcagttgcagaccagagagccgccttcgccactggtgttcttccatatatctacgcattccaccgctacacatggagttccactctcctcttctgcactcaagttcaacagtttctgatgcaattctccggttgagccgaaggctttcacatcagacttattgaaccgcctgcactcgctttacgcccaataaatccggacaacgcttgccacctacgtattaccgcggctgctggcacgtagttagccgtgactttctaagtaattaccgtcaaataaaggccagttactacctctatctttcttcactaccaacagagctttacgagccgaaacccttcttcactcacgcggcgttgctccatcagactttcgtccattgtggaagattccctactgctgcctcccgtaggagtttgggccgtgtctcagtcccaatgtggccgatcagtctctcaactcggctatgcatcattgccttggtaagccgttaccttaccaactagctaatgcaccgcaggtccatccaagagtgatagcagaaccatctttcaaactctagacatgcgtctagtgttgttatccggtattagcatctgtttccaggtgttatcccagtctcttgggcaggttacccacgtgttactcacccgtccgccgctcgcttgtatctagtttcatttagtgcaagcactaaaatcatctaggcaagctcgctcgacttgcatgtattaggcacgccgccagcgttcgtcctgagccatggatcaa

SEQ ID NO: 2 - DNA, 896 bp, Lactobacillus johnsonii
accttctggcaattcaatcttaccagttacgtcagtagtgtggaagtagaattgtggacggtaatctgagaagaatggagtgtgacgaccaccttcatctttgttcaagatataaacttgacccttgaagttcttgtgggtttgaattgaaccaggtgcagctaaaacttgaccacgttcaacttgatcacggtcgataccacgaagcaatacaccaacgttatcgccggcttcaccaaggtcaagagtcttgtggaacatttccaaaccagtaacagttgacttttcaatcttgtcagttaaaccaacgatttcaacttcatcgccgaccttaacagtaccacggtcgatacgaccagaagcaacagtaccacgaccagtgatagtaaatacgtcttcaactggcattaagaatggcttgtcagtatcacgttctggagttgggatgtattcgtcaacagtttccattaattttctgataacgtcttgttgttctgggtcaccttcaagtgccttcaaagctgaaccacggataacaggaacatcgtcaccagggtaatcgtattctgataacaagtcacgtacttccatttcaactaagtcgatcaattctggatcgtcaactaagtcaaccttgtttaagaatacaacgatgtattgaacaccaacttgacgagcaagtaagatgtgttcacgagtttgtggcataggaccatcagttgcagcaacaaccaagatagcaccatccatttgtgcagcaccagtaatcatgttcttgatgtagtcagcgtgacctggagcatccatgtgagcgtagtgacgattcttagtttcgtattctacgtgagcggtattaatagtaataccacgttccttttcttctgggagcagcatca